(19) United States
(12) Patent Application Publication
(10) Pub. No.: US 2010/0015689 A1
(43) Pub. Date: Jan. 21, 2010

(54) Method For Composting And Treating Food Waste By Using Wood Chips And Apparatus Therefor

(75) Inventors: Wan Cheol Park, Seoul (KR); Tae Hyung Kim, Seongnam-si (KR); Mi Ae Lee, Seoul (KR)

Correspondence Address: JONES DAY, 222 EAST 41ST ST, NEW YORK, NY, 10017, US

(73) Assignee: Korea Institute of Science and Technology

(21) Appl. No.: 12/502,990

(22) Filed: Jul. 14, 2009

(30) Foreign Application Priority Data
Jul. 17, 2008 (KR) 10-2008-0069523

(51) Int. Cl. C12S 3/00; C12M 1/04

(52) U.S. Cl. 435/267; 435/290.4

(57) ABSTRACT

The present invention relates to a method of composting and treating food waste by using wood chips and an apparatus therefor. The method and apparatus for the composting and treatment of food waste in accordance with the present invention can recycle food waste as an organic compost by using wood chips in an eco-friendly manner and convert effluents generated from said food waste into an effluent satisfying water quality suitable for discharging by a combined biological and chemical process. Therefore, the present invention can be effectively used for recycling and treating food waste.

The present application claims priority from Korean Patent Application No. 10-2008-69523 filed Jul. 17, 2008, the subject matter of which is incorporated herein by reference.

### FIELD OF THE INVENTION

The present invention relates to a method of composting and treating food waste by using wood chips and an apparatus therefor.

### BACKGROUND OF THE INVENTION

More and more households and restaurants are generating large amounts of leftover food. Some of the leftover food is used as feed for domestic animals, but due to the difficulty in containing, transporting and/or treating the leftover food, almost all leftover food/food waste is reclaimed or incinerated.

In Korea, food waste corresponding to 8 trillion won is generated every year. Although such food waste may be converted to reusable resources, such treatment is not easy. According to statistical data from the Ministry of Environment for the year 2000, food waste reused as feedstuff and compost amounted to 5,600 tons of total food waste of 11,350 tons, where the reuse ratio of food waste reached 49.3%. Generally, since most food waste consists of organic materials, problems relating to the stabilization of soil and landfill leachate may be caused when such waste is buried.

Reclamation methods typically produce foul odors which are generated by nitrogen and sulfur compounds released during the process. In addition, reclamation methods generate a high density effluent that contaminates the atmosphere, water and soil.

Incineration requires incinerators of high capacity and contaminates the atmosphere by releasing harmful substances into the air. Furthermore, the efficiency of incinerating leftover food is decreased by low-caloric leftover food and also by moisture in the leftover food. Therefore, in addition to also releasing dangerous contaminants (like dioxin), this process can also be expensive.

Another method of removing leftover food waste is the dry method system. The dry method system dehydrates food waste by stirring and chopping dried food waste. However, this method is not widely practiced by regular households due to the substantial cost of electricity.

In addition, there is a method of degrading the collected food waste via anaerobic digestion, where a form of a septic tank in a small apartment area and a municipal plant is used. This method is being used increasingly.

In this regard, Korean Patent No. 0280934 discloses a simultaneous disposal method for ordure, food waste and domestic sewage. Additionally, Korean Patent No. 0266089 discloses a technique of treating organic materials constituting food waste, using aerobic microorganisms, water, air and hydrogen peroxides.

However, the above methods are disadvantageous in that, since the transported food waste is treated together with the domestic sewage and not separated, the sewage and food waste generate high loads on the disposal facility, thus requiring a disposal facility of a large capacity.

Yet another method of removing leftover food waste is by decomposition. Decomposition methods provide an optimal environment for microorganisms to grow and decompose food waste into H₂O and CO₂. These systems sometimes require stirring equipment to mix the food waste in a processing container. In certain situations, such a system may require a separate chopper in order to process tough or hard food waste. Therefore, this system is not always suitable for a small scale operation. In addition, flexible and long materials are often not capable of being cut by a blade and can end up being wrapped around the stirring axis of the container. When food waste becomes wrapped around a stirring axis, the motor may become overloaded and result in a malfunction or fire.

Additionally, these decomposition methods still yield large amounts of decomposed waste material or sediment. For example, composting may result in only about a 45% decrease in waste material mass—still leaving a significant amount of sediment behind. While the remaining material or sediment can be used as compost, such large amounts of remaining mass may not be practical for commercial and urban uses.

Therefore, there is a strong need to find alternative approaches for reducing, treating and recycling food waste. Despite such need, no satisfactory approaches have been introduced so far.

### SUMMARY OF THE INVENTION

One of the objectives of the present invention is to provide a method of composting and treating food waste which can recycle a high concentration of organic materials included in food waste as organic compost and convert effluents generated from the food waste into an effluent satisfying water quality suitable for discharging by a biological and chemical combined treatment, and an apparatus therefor.

In order to achieve the above objective, one embodiment of the present invention relates to a method of composting and treating food waste by using wood chips, which involves:

separating organic materials from food waste by absorbing to wood chips and fermenting the same, and

treating effluents generated from said food waste by a combined biological and chemical process using aerobic microorganisms.

Another embodiment of the present invention relates to an apparatus for composting and treating food waste according to the above method.

### BRIEF DESCRIPTION OF THE DRAWING

The embodiments of the present invention will be described in detail with reference to the following drawing.

FIG. 1 is a diagram schematically illustrating an apparatus for the composting and treatment of food waste by using wood chips according to the present invention.

1: first wood chip absorbing tank; 2: second wood chip absorbing tank;
3: air floatation tank; 4: anaerobic tank; 5: microbial activation tank; 6: aeration tank;
7: denitrification tank; 8: precipitation tank; 9: chemical treatment tank;
10: composting tank; 11, 12, 13, 14 and 15: mixers;
16: first inflow line of wood chips into a composting tank;
17: first return line of sludge from a denitrification tank to an anaerobic tank;
18: second return line of sludge from a precipitation tank to a composting tank;
19: second return line of sludge from a precipitation tank to an anaerobic tank;
20: second return line of sludge from a precipitation tank to a wood chip absorbing tank;
21: second return line of sludge from a precipitation tank to a microbial activation tank;
22: second inflow line of sludge from an air floatation tank to a composting tank;
23: waste sludge line for discharging sludge from the precipitation tank;
24: liquefied fertilizer collection line;
25: third inflow line of short-chain organic acids from a wood chip absorbing tank to a denitrification tank

### DETAILED DESCRIPTION OF THE INVENTION

The present invention provides a method of composting and treating food waste by using wood chips, which involves:

separating organic materials from food waste by absorbing to wood chips and fermenting the same, and

treating effluents generated from said food waste by a combined biological and chemical process using aerobic microorganisms.

The method according to the present invention is characterized in that solid organic materials contained in food waste at a high concentration are separated by absorbing to wood chips and subjected to composting by fermenting said wood chips, and effluents generated from said food waste whose organic materials are separated are converted into an effluent satisfying water quality suitable for discharging by a combined biological and chemical treatment.

In particular, the method of composting and treating food waste according to the present invention involves the following steps:
(1) mixing food waste with wood chips and separating solid organic materials from food waste by absorbing the same to wood chips;
(2) collecting the wood chips to which the organic materials are absorbed in step (1) and fermenting the same under aerobic conditions, to thereby obtain an organic compost;
(3) treating effluents generated from said food waste after the organic materials are separated therefrom in step (1) under pressure, to thereby move untreated suspended particles to an upper part and move a supernatant in which organic materials are dissolved to a lower part;
(4) transferring the suspended particles which have passed through step (3) to step (2) and fermenting the same to obtain an organic compost, while denitrifying nitrate nitrogen (NOx-N) included in sludge, which is fed through a return line in the following steps (6) and (7), by using the organic materials remaining in the resulting supernatant which has passed through step (3) as a carbon source;
(5) degrading the organic materials remaining in the resulting material which has passed through step (4) under aerobic conditions by using an aerobic microorganism and oxygen;
(6) denitrifying nitrate nitrogen included in the resulting material which has passed through step (5) by using short-chain organic acids as a carbon source;
(7) separating the resulting material which has passed through step (6) into a precipitated sludge and a supernatant; and
(8) transferring the precipitated sludge which has passed through step (7) to step (4) through a return line and chemically treating the supernatant which has passed through step (7) to remove non-biodegradable materials.

Further, the present invention provides an apparatus for composting and treating food waste, including:
first and second wood chip absorbing tanks where organic materials included in food waste are separated from food waste by absorbing to wood chips;
a composting tank where the wood chips to which organic materials are absorbed are fermented under aerobic conditions, to thereby obtain organic compost;
an air floating tank where effluents generated from said food waste in the wood chip absorbing tanks are treated under pressure so as to separate suspended particles and a supernatant;
an anaerobic tank where nitrate nitrogen (NOx-N) included in sludge, which is fed through a return line, is subjected to denitrification by using the organic materials remaining in the resulting supernatant passed through the air floating tank as a carbon source;
a microbial activation tank where aerobic microorganisms suitable for the removal of organic materials are activated;
an aeration tank where organic materials and nitrogen remaining in the resulting material passed through the anaerobic tank is degraded by using the aerobic microorganisms activated in the microbial activation tank under aerobic conditions;
a denitrification tank where nitrate nitrogen remaining in the resulting material passed through the aeration tank is subjected to denitrification by the action of denitrifying microorganisms;
a precipitation tank where the resulting material passed through the denitrification tank is separated into a precipitated sludge and a supernatant; and
a chemical treatment tank where the supernatant separated in the precipitation tank is chemically treated to remove non-biodegradable materials.

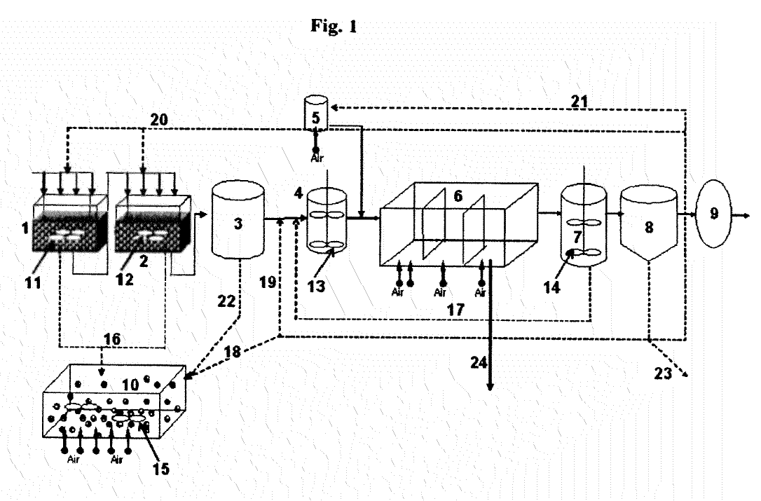

A representative example of the apparatus according to the present invention is described in FIG. 1. Referring to FIG. 1, the apparatus for composting and treating food waste in accordance with the present invention may include:
first and second wood chip absorbing tanks 1,2 for efficiently absorbing and separating organic materials included in food waste at a high concentration by using wood chips;
a composting tank 10 for fermenting the wood chips to which organic materials are absorbed into organic compost;
an air floatation tank 3 for separating effluents generated from said food waste whose organic materials are removed in the wood chip absorbing tanks into suspended particles and a supernatant containing organic materials;
an anaerobic tank 4 for denitrifying nitrate nitrogen included in sludge which is fed through a return line;
a microbial activation tank 5 for activating aerobic microorganisms suitable for the removal of organic materials;
an aeration tank 6 for degrading organic materials and nitrogen remaining in the resulting material by using aerobic microorganisms and oxygen;
a denitrification tank 7 for denitrifying nitrate nitrogen degraded in the aeration tank 6 by the action of denitrifying microorganisms;
a precipitation tank 8 for separating the resulting material into a precipitated sludge and a supernatant by means of gravity;
a chemical treatment tank 9 for treating the supernatant and removing non-biodegradable materials therefrom;
mixers 11,12,13,14,15 for mixing the resulting material in each of the first and second wood chip absorbing tanks 1,2, anaerobic tank 4, denitrification tank 7 and composting tank 10;
a first inflow line 16 for feeding the wood chips to which organic materials are absorbed passed through the first and second wood chip absorbing tanks 1,2 into the composting tank 10;
a first return line 17 for transferring sludge from the denitrification tank 7 to the anaerobic tank 4;
one or more second return lines 18,19,20,21 for transferring sludge from the precipitation tank 8 to the composting tank 10, anaerobic tank 4, first and second wood chip absorbing tanks 1,2 and microbial activation tank 5, respectively;
a second inflow line 22 for feeding sludge from the air floatation tank 3 to the composting tank 10;
a waste sludge line 23 for discharging sludge from the precipitation tank 8;
a liquefied fertilizer collection line 24 for utilizing the resulting material stabilized in the aeration tank 6 as a liquefied fertilizer; and
a third inflow line 25 for feeding short chain organic acids from the second wood chip absorbing tank 2 into the denitrification tank 7.

Hereinafter, the present invention will be explained in more detail.

In the method of composting and treating food waste according to the present invention, step (1) is a process for mixing food waste with wood chips and separating solid organic materials included in said food waste at a high concentration by absorbing the same to wood chips.

Food waste is mainly composed of solid state organic materials such as carbohydrates, fats, proteins and the like. Thus, it requires a relatively long period of time for hydrolysis due to the various particle sizes and types of the constituents. For the efficient composting and treatment of such food waste, the present invention has developed a method of using wood chips.

The term “wood chips” as used herein refers to wood fragments made by finely pulverizing waste wood byproducts such as fallen wood, thinned wood, pruned wood, bark, sawdust and the like. Wood chips facilitate the supply of oxygen by forming a porous structure within the mixture. Further, since they absorb a lot of moisture while water is supplied and emit moisture gradually thereafter, wood chips can smoothly supply moisture essential for the proliferation and growth of microorganisms. In addition, wood chips are served as a medium to which microorganisms adhere, and thus, create a favorable environment for the proliferation and growth of microorganisms, which leads to the successful composting of food waste. As suitable timber for wood chips, it is desirable to use coniferous trees resistant to microbial degradation, more specifically, cypress, cedar, pine, fir, spruce and the like. In particular, cypress (Crysptomeria sp.) which has a well developed fibrous structure and is inexpensive is desirable timber that can be used as wood chips. In some embodiments, the present invention prepares wood chips by pulverizing domestically produced cypress. Here, the thus prepared wood chips have an average particle size in the range of 2 to 5 cm.

First, food waste is fed into a first wood chip absorbing tank 1 which is filled with fresh wood chips with an equal amount of recycled wood chips returned from a composting tank 10. When the hydraulic retention time (HRT) of the resulting material in the absorbing tank is considered, food waste is specifically fed into the first wood chip absorbing tank 1 in an amount ranging from 58 to 70 kg/l per day, and is mixed with wood chips in a weight ratio ranging from 45:55 to 55:45. The first wood chip absorbing tank 1 is equipped with a mixer 11 so as to homogeneously mix food waste and wood chips, thereby efficiently absorbing organic materials in food waste to wood chips. The hydraulic retention time (HRT) of the resulting material in the first wood chip absorbing tank 1 is specifically 20 to 24 hours. The resulting material passed through the first wood chip absorbing tank 1 is transferred to a second wood chip absorbing tank 2. In the second wood chip absorbing tank 2, fresh wood chips are filled with an equal amount of recycled wood chips returned from a composting tank 10, and a mixer 12 is installed so as to homogeneously mix food waste and wood chips, thereby efficiently absorbing organic materials in food waste to wood chips. The hydraulic retention time (HRT) of the resulting material in the second wood chip absorbing tank 2 is specifically 20 to 24 hours.

Step (2) is a process for collecting the entire quantity of wood chips to which organic materials are absorbed from the first and second wood chip absorbing tanks 1,2 and fermenting the same under aerobic conditions, to thereby obtain organic compost.

After passing through the first and second wood chip absorbing tanks 1,2 in step (1), the solid organic materials included in food waste at a high concentration are absorbed to wood chips. The wood chips are completely recovered from the absorbing tanks and transferred to a composting tank 10 through a first return line 16. In the composting tank 10, the wood chips are mixed with oxygen by means of a mixer 15, and then, are subjected to a composting process via aerobic fermentation. It is desirable to dry the above wood chips and maintain low moisture content therein before transferring to the composting tank 10, while the aerobic fermentation is specifically carried out at a temperature ranging from 50 to 70° C. for 3 to 5 days. To the composting tank 10, a fixed amount of sludge activated in a precipitation tank 8 is fed through a second return line 18, which provides activated microorganisms for the aerobic fermentation and to remove foul odor generated during the fermentation in the composting tank 10 by the action of the microorganisms.

When the composted mixture reaches a stable state as organic materials are oxidized, the wood chips which have passed through the aerobic fermentation are separated from the composted mixture via a sorting process. The thus separated wood chips are transferred to the absorbing tanks and reused, while the remaining material is used as organic compost. The above sorting process is carried out by passing the composted mixture of wood chips and food waste through a sorter, thereby separating the organic compost and wood chips by filtering. The sorter used in this step can be appropriately selected from those conventionally used in the preparation of a byproduct fertilizer or compost. In some embodiments, the present invention employs a sorter in which three types of sieves having pore sizes of 10 mm, 8 mm and 6 mm, respectively, are arranged at regular intervals. When the composted mixture is passed through the sorter, wood chips and impurities are filtered by these sieves, while pure organic compost is collected. The thus separated wood chips to which a large quantity of activated microorganisms are adhered are returned to the first and second wood chip absorbing tanks 1,2. Here, the amount of wood chips returned is specifically a half percent of the total weight of wood chips filled in each tank. Such recycling of the wood chips can expect to reduce operating costs and remove foul odor generated from the absorbing tank.

Composting is a process of degrading organic materials by the action of microorganisms and stabilizing. Compost is generally recommended as an additive to soil supplying humus and nutrients. It provides a rich growing medium, or a porous, absorbent material that holds moisture and soluble minerals, providing support and nutrients. The degradation mechanisms of carbohydrates, proteins and fats in the composting process are different each other. First, carbohydrates are converted into monohydrates, which are reacted with oxygen, thereby degrading into carbon dioxide and water. The degradation mechanism of carbohydrates which is identical to combustion is performed at a relatively low temperature by using oxygen as a catalyst. Generally, the chemical equation for the degradation of carbohydrates is as follows:

Cₘ(H₂O)ₙ+m(O₂)→mCO₂+nH₂O

If oxygen is lacking in the composting process, anaerobic conditions are made, and thereby, organic acids are generated, leading to a lowering of the pH.

Proteins and fats degrade into small molecules having a low molecular weight while generating carbon dioxide, water and ammonia. The chemical equation for the degradation of proteins and fats is as follows:

CₓH_{y}N_{z}Oₚ+aO₂→CᵤHᵥNₘO_{q}+bCO₂+dH₂+eNH₃

In the above reaction, ammonia (NH₃) is dissolved in water and converts into NH₃OH, thereby increasing the pH.

Step (3) is a process for treating effluents generated from the food waste after its organic materials are separated in the first and second wood chip absorbing tanks 1,2 of step (1) under pressure, to thereby move untreated suspended particles to an upper part and move a supernatant in which organic materials are dissolved to a lower part.

Although the resulting material passed through the first and second wood chip absorbing tanks 1,2 shows a significantly reduced concentration of suspended particles, its concentration is still high to be introduced into a biological treatment apparatus as it is. If the resulting material having a high concentration of suspended particles is introduced into an aeration tank 6, it is difficult to maintain a proper concentration of mixed liquor suspended solids (MLSS) in the aeration tank. Therefore, in order to maintain a proper concentration of MLSS, there is a need to decrease the concentration of suspended particles in the resulting material, and thereby, reduce the load of suspended particles in the aeration tank. In order to accomplish this, after the resulting material which has passed through the first and second wood chip absorbing tanks 1,2 is transferred to the air floatation tank 3, compressed air is supplied thereto, and thereby, the suspended particles float up to a upper part while moving down a supernatant in which organic materials are dissolved to a lower part. The thus floated suspended particles are transferred to the composting tank 10 through a second inflow line 22 and subjected to the composting process of step (2), while the remaining supernatant containing organic materials is transferred to an anaerobic tank 4 which is a biological treatment apparatus.

Step (4) is a process for denitrifying nitrate nitrogen (NOx-N) included in sludge which is fed through a return line in the following steps (6) and (7) by using the organic materials remaining in the supernatant which have passed through the air floatation tank 3 in step (3) as a carbon source.

The sludge precipitated in the denitrification tank 7 and precipitation tank 8 is transferred to the anaerobic tank 4 through second return lines 18,19, respectively. The nitrate nitrogen (NOₓ—N) included in the returned sludge is subjected to denitrification under anaerobic conditions by using the organic materials included in the supernatant transferred from the air floatation tank 3 as a carbon source. Through the above denitrification, nitrogen is removed and phosphorous is eluted. When there is little dissolved oxygen as in the anaerobic tank 4, energy is obtained by using a nitrogen-oxygen complex in the form of NO₃ and NO₂ as an electron acceptor. Further, nitrate nitrogen (NOₓ—N) is converted into nitrogen gas (N₂) by the action of denitrifying microorganisms (denitrifiers) and released into the air, as follows:

NO₃→NO₂→NO→N₂O→N₂

Further, the anaerobic tank 4 may be equipped with a mixer 12 so as to homogeneously mix the denitrifying microorganisms, carbon source and nitrate nitrogen. Since organic acids used as an electron donor in the denitrification process are plentiful in the resulting material passed through the first and second wood chip absorbing tanks 1,2, said denitrification process according to the present invention can be effectively performed without adding an external carbon source.

The step (5) is a process for degrading the organic materials remaining in the resulting material passed through the step (4) under aerobic conditions by using aerobic microorganisms and oxygen.

In order to use microorganisms showing high removal efficiencies for odor and organic materials in the treatment of food waste, the present invention have isolated such microorganisms having these characteristics selectively from soil, cultured them in large quantities, and then, formulated into a microbial colony. The microbial colony useful for the present invention is composed of ingredients effective for the proliferation and activation of anaerobic microorganisms such as Bacillus species in the anaerobic treatment and composting process. Generally, Bacillus species is known as a microorganism ubiquitous in nature which can survive under stressful environmental conditions and exhibits high efficiencies for the degradation of organic materials and denitrification. Food waste has a pH range between 4 and 6, which is significantly lower than other waste materials, and shows a considerably high salt concentration. Such stressful environmental conditions of food waste make remarkably reduced the viability and activity of common microorganisms. However, Bacillus species can survive under such a low pH range and high salt concentration and exhibit its biological functions including the degradation of organic materials and denitrification.

The thus prepared microbial colony is activated under aerobic conditions in the microbial activation tank 5, and then, transferred to an aeration tank 6 through an inflow line. The inside of the activation tank 5 is kept under aerobic conditions by introducing external air, thereby selectively activating the aerobic microorganisms. Further, the activation tank 5 is designed to continuously supply sludge through a second return line 21, which results in the successive activation of microorganisms eluted from the microorganism colony and those included in the returned sludge.

The organic materials included in the resulting material which has passed through the denitrification step are degraded in the aeration tank 6 under aerobic conditions by using the activated aerobic microorganisms and oxygen.

The term “aerobic degradation” used herein refers to a process in which aerobic microorganisms take in organic materials included in food waste, use them as a nutrient source for survival, and then degrade them into carbon dioxide, ammonia, water and the like. The produced ammonia in such a process binds to oxygen and is oxidized into nitrate (NO₃⁻) nitrogen by way of nitrite (NO₂⁻) nitrogen. Such an aerobic degradation takes place in the aeration tank 6 of the apparatus adapted to treat food waste in accordance with the present invention. Here, the aerobic degradation of the present invention is specifically carried out at a temperature of 25 to 35° C. for 10 to 15 hours, and the concentration of MLSS in the aeration tank 6 is specifically maintained in the range of 9,000 to 12,000 mg/l.

The aeration tank 6 is divided into a number of compartments by means of a plurality of diaphragms. The amount of dissolved oxygen (DO) in each compartment is differently maintained in a graduated manner. In some embodiments of the present invention, the aeration tank 6 may be divided into three compartments. Here, a first compartment of the aeration tank is designed to effectively degrade organic materials in food waste by maintaining a high DO concentration through the inflow of external air. Further, ammonia nitrogen is oxidized into nitrate (NO₃—) nitrogen via nitrite (NO₂⁻) nitrogen by the action of denitrifying microorganisms in this compartment. In a third compartment of the aeration tank, the supply of oxygen is discontinued so as to maintain a low DO concentration. It is desirable that the DO concentration of the first compartment is maintained in the range of 0.5 to 1.0 mg/l, while that of the second compartment is maintained in the range of 0.2 to 0.5 mg/l. Further, it is desirable that the DO concentration of the third compartment is maintained at 0.1 mg/l or below. The aeration tank 6 is equipped with a collection line 24 of a liquefied fertilizer for utilizing the stabilized liquid in the aeration tank as a liquefied fertilizer.

Step (6) is a process for denitrifying nitrate nitrogen (NOₓ—N) remaining in the resulting material which has passed through the aerobic degradation step by using short-chain organic acids as a carbon source, which is fed into a denitrification tank 7 through an inflow line. The denitrification is specifically carried out at a temperature of 25 to 35° C. for 3 to 6 hours.

The resulting material which has passed through the aerobic degradation in the aeration tank 6 is transferred to the denitrification tank 7. Under anaerobic conditions, nitrate nitrogen is used as an oxygen source for denitrifying microorganisms and is converted into N₂ gas (said process is referred to as denitrification). The denitrification process takes place in the denitrification tank 7 of the apparatus adapted to treat food waste in accordance with the present invention. The denitrification efficiency can be maximized by using short-chain organic acids introduced from the second wood chip absorbing tank 2 into the denitrification tank 7 though a third inflow line 25 as a carbon source. The resulting material which has passed through the denitrification tank 7 is transferred to a precipitation tank 8.

Step (7) is a process for separating the resulting material which has passed through the denitrification step into a precipitated sludge and a supernatant.

The above process is carried out in the precipitation tank 8 of the apparatus adapted to treat food waste in accordance with the present invention. The food waste treated in the denitrification step is subjected to solid-liquid separation by means of gravity, thereby being separated into a precipitated sludge and a supernatant. The separated supernatant is transferred to the next step for chemical treatment. On the other hand, a part of the precipitated sludge is transferred to the anaerobic tank 4 through a second return line 19, while nitrate nitrogen included therein is subjected to denitrification. Some other parts of the precipitated sludge are transferred to the composting tank 10 through a second return line 18 so as to provide aerobic microorganisms and remove foul odor, transferred to the first and second wood chip absorbing tanks 1,2, and transferred to the activation tank 5 through a second return line 21 so as to promote the activation of aerobic microorganisms, respectively. The rest goes into disuse through a waste slude line 23.

Step (8) is a process for transferring the precipitated sludge separated in the above step to each reaction tank through a return line and chemically treating the supernatant to remove non-biodegradable materials.

The above process takes place in the chemical treatment tank 9 of the apparatus adapted to treat food waste in accordance with the present invention. In this process, non-biodegradable materials such as phosphorus, suspended materials, color inducing materials and the like can be removed by adding a coagulant. The coagulant employable in the present invention may include Al-based coagulants, Fe-based coagulants and the like, but is certainly not limited thereto. Representative examples of the Al-based coagulant may include Alum (Al₂SO₄).18H₂O, PAC (poly aluminum chloride) and the like. Those of the Fe-based coagulants may include FeSO₄.7H₂O, FeCl₃, Fe₂(SO₄)₃ and the like. Chemical treatment is carried out by considering the effects of the type and inflow amount of coagulant, pH, turbidity and the like. Further, a coagulant aid may be further employed in this step. Representative examples of the coagulant aid may include a pH regulator such as calcium hydroxide (Ca(OH)₂), calcium oxide (CaO), sodium hydroxide (NaOH) and sodium carbonate (Na₂CO₃), as well as a turbidity enhancer such as bentonite, flay ash, activated silica and cement dust. As described above, the present invention can recycle food waste as organic compost by using wood chips in an eco-friendly manner and convert effluents generated from said food waste into an effluent satisfying water quality suitable for discharging by a combined biological and chemical process. Therefore, the present invention can be effectively used for recycling and treating food waste.

### EXAMPLES

Embodiments of the present invention will now be described in more detail with reference to the following examples. However, the examples are provided for purposes of illustration and are not to be construed as limiting the scope of the invention.

### Example 1

Each reactor used in the apparatus for composting and treating food waste according to the present invention was manufactured from acryl so as to easily allow the observation of the interior of the reactor.

Table 1 shows the resource and size of each reactor in the apparatus of the present invention. The return line in the apparatus of the present invention was equipped with a transfer pump. Further, an aeration tank and a microbial activation tank were equipped with a ventilator.

TABLE 1

| Reactor and device | Size | Remarks |
|---|---|---|
| First wood chip | 70 l (L = 50 cm, W = 40 cm, | Rectangular |
| absorbing tank | H = 40 cm) | |
| Second wood chip | 70 l (L = 50 cm, W = 40 cm, | Rectangular |
| absorbing tank | H = 40 cm) | |
| Anaerobic tank | 5 l (d = 16 cm, H = 35 cm) | Cylindrical |
| Aeration tank | 10 l (L = 33 cm, B = 17 cm, | Rectangular, sewage sludge |
| | H = 27 cm) | |
| Denitrification tank | 3 l (d = 14 cm, H = 30 cm) | Cylindrical |
| Precipitation tank | 4 l (d = 23 cm, H = 35 cm) | Conical, hopper slope 60° |
| | | maintenance |
| Microbial activation tank | 0.2 l (d = 0.7 cm, H = 18 cm) | Cylindrical |
| pH, temperature controller | Orione 250A | pH, temperature measurement |
| Transfer pump | Master-flex pump | 2 heads |
| Mixer | Panasonic M6GA30M | 60 rpm |
| Ventilator | Techno Takasuki Co., Ltd | 40 l/min capacity |
| | SPP-200GJ-H | |

Food waste used in the following experiment was collected from the refectory of Korea Institute of Science and Technology, finely pulverized with a blender, and stored in a cold chamber until use.

The HRT of each reactor was regulated to be 20 to 24 hours for each of the first and second wood chip absorbing tanks; 3 to 6 hours for the anaerobic tank; 3 days for the microbial activation tank; 3 to 6 hours for the denitrification tank; and 8 hours for the precipitation tank. During the chemical treatment, ferric chloride (FeCl₃) was employed as a coagulant at a concentration of 100 mgFe/l.

The MLSS concentration of the aeration tank was maintained at 10,000 mg/l. For maintaining such a MLSS concentration, a certain amount of surplus sludge was discarded from the precipitation tank. In addition, the apparatus was configured to transfer the object to be treated by a natural flowing method using gravity. The input and transfer of a sample were automatically controlled by using a quantitative pump equipped with a timer so as to ensure its accurate amount.

After treating food waste by using the apparatus of the present invention, chemical oxygen demand (CODcr: determined using potassium bichromate(K₂Cr₂O₇) as an oxidizing agent), biological oxygen demand (BOD), total suspended solids (TSS), total Kjeldahl nitrogen (TKN), NH₄ and the total amount of phosphorus (T-P) of effluents obtained in each step were measured, as shown in Table 2. At this time, the experimental data were represented as a mean value during the operation.

TABLE 2

| | CODcr | BOD | TSS | TKN | NH₄ | NOx—N | T-P |
|---|---|---|---|---|---|---|---|
| | (mg/l) | (mg/l) | (mg/l) | (mg/l) | (mg/l) | (mg/l) | (mg/l) |
| Raw water | 311,400 | 118,200 | 209,600 | 9,220 | 166 | 0 | 1,138 |
| Effluent from first wood | 81,766 | 45,120 | 29,845 | 2,823 | 384 | 16 | 459 |
| chip absorbing tank | | | | | | | |
| Effluent from second | 63,323 | 34,200 | 18,360 | 2,266 | 412 | 7 | 349 |
| wood chip absorbing tank | | | | | | | |
| Air floatation tank | 29,500 | 12,500 | 9,800 | 2,150 | 382 | 5 | 250 |
| Biologically treated | 1,082 | 55 | 72 | 69 | 55 | 1.0 | 52 |
| effluent | | | | | | | |
| Chemically treated | 51 | 17 | 15 | 43 | 35 | 1.0 | 4 |
| effluent | | | | | | | |

As described in Table 2 above, the properties of raw water of the pulverized food waste were as follows: TSS concentration was 209,600 mg/l; CODcr concentration was 311,400 mg/l; BOD concentration was 118,200 mg/l; and T-P concentration was 1,138 mg/l. Further, TKN concentration was 9,220 mg/l and NH₄ concentration was 166 mg/l.

The ammonia concentration of the effluent obtained from the first and second wood chip absorbing tanks was significantly high compared to that of the raw water, which is because organic nitrogen was subjected to ammonification in the absorbing tanks. The water quality of the effluent obtained from the precipitation tank, which was regarded as a biological treatment process, was as follows: CODcr concentration was 1,082 mg/l; BOD concentration was 55 mg/l; TSS concentration was 72 mg/l; TKN concentration was 69 mg/l; NH₄ concentration was 55 mg/l; and T-P concentration was 52 mg/l. The water quality of the effluent obtained after the chemical treatment was as follows: CODcr concentration was 51 mg/l; BOD concentration was 17 mg/l; TSS concentration was 15 mg/l; TKN concentration was 43 mg/l; NH₄ concentration was 35 mg/l; and TP concentration was 4 mg/l.

It was confirmed that as the steps for treating food waste progress, the water quality of effluents is improved to a level sufficient enough for discharge.

While the present invention has been described and illustrated with respect to a preferred embodiment of the invention, it will be apparent to those skilled in the art that variations and modifications are possible without deviating from the broad principles and teachings of the present invention, which should be limited solely by the scope of the claims appended hereto.

What is claimed:

1. A method of composting and treating food waste comprising:
separating organic materials in food waste by absorbing to wood chips and fermenting the same, and
treating effluents generated from said food waste by a combined biological and chemical process using aerobic microorganisms.

2. The method according to claim 1, which comprises:
(1) mixing food waste with wood chips and separating solid organic materials from food waste by absorbing the same to wood chips;
(2) collecting the wood chips to which the organic materials are absorbed from step (1) and fermenting the same under aerobic conditions, to thereby obtain an organic compost;
(3) treating effluents generated from said food waste after the organic materials are separated therefrom in step (1) under pressure, to thereby move untreated suspended particles to an upper part and move a supernatant in which organic materials are dissolved to a lower part;
(4) transferring the suspended particles which have passed through step (3) to step (2) and fermenting the same to obtain an organic compost, while denitrifying nitrate nitrogen (NOx-N) included in sludge, which is fed through a return line in the following steps (6) and (7), by using the organic materials remaining in the resulting supernatant which has passed through step (3) as a carbon source;
(5) degrading the organic materials remaining in the resulting material which has passed through step (4) under aerobic conditions by using an aerobic microorganism and oxygen;
(6) denitrifying nitrate nitrogen included in the resulting material which has passed through step (5) by using short-chain organic acids as a carbon source;
(7) separating the resulting material which has passed through step (6) into a precipitated sludge and a supernatant; and
(8) transferring the precipitated sludge which has passed through step (7) to step (4) through a return line and chemically treating the supernatant which has passed through step (7) to remove non-biodegradable materials.

3. The method according to claim 2, wherein the wood chips in step (1) have an average size in the range of 2 to 5 cm.

4. The method according to claim 2, wherein the wood chips in step (1) is a mixture of equal amounts of fresh wood chips and recycled wood chips which have passed though the fermenting process in step (2).

5. The method according to claim 2, wherein the wood chips in step (1) is mixed with food waste in a weight ratio of 45:55 to 55:45.

6. The method according to claim 2, wherein the fermentation process in step (2) is carried out at a temperature of 50 to 70° C. for 3 to 5 days.

7. The method according to claim 2, wherein the denitrification process in step (4) is carried out at a temperature of 25 to 35° C. for 3 to 6 hours.

8. The method according to claim 2, wherein the aerobic degradation process in step (5) is carried out at a temperature of 25 to 35° C. for 10 to 15 hours.

9. The method according to claim 2, wherein the denitrification process in step (6) is carried out at a temperature of 25 to 35° C. for 3 to 6 hours.

10. The method according to claim 2, wherein the chemical treatment process in step (8) is carried out in the presence of a coagulant.

11. The method according to claim 10, wherein the coagulant is an aluminum (Al)-based or an iron (Fe)-based coagulant.

12. An apparatus for compositing and treating food waste, comprising:
first and second wood chip absorbing tanks where organic materials included in food waste are separated from food waste by absorbing to wood chips;
a composting tank where the wood chips to which organic materials are absorbed are fermented under aerobic condition, to thereby obtain an organic compost;
an air floating tank where effluents generated from said food waste in the wood chip absorbing tanks are treated under pressure so as to separate suspended particles and a supernatant;
an anaerobic tank where nitrate nitrogen (NOx-N) included in sludge, which is fed through a return line, is subjected to denitrification by using the organic materials remaining in the resulting supernatant which has passed through the air floating tank as a carbon source;
a microbial activation tank where aerobic microorganisms suitable for the removal of organic materials are activated;
an aeration tank where organic materials and nitrogen remaining in the resulting material which has passed through the anaerobic tank is degraded by using the aerobic microorganisms activated in the microbial activation tank under aerobic conditions;
a denitrification tank where nitrate nitrogen remaining in the resulting material which has passed through the aeration tank is subjected to denitrification by the action of denitrifying microorganisms;
a precipitation tank where the resulting material which has passed through the denitrification tank is separated into a precipitated sludge and a supernatant; and
a chemical treatment tank where the supernatant separated in the precipitation tank is chemically treated to remove non-biodegradable materials.

13. The apparatus according to claim 12, further comprising:
mixers for homogeneously mixing the resulting material in each of the first and second wood chip absorbing tanks, anaerobic tank, denitrification tank and composting tank;
a first inflow line for feeding the wood chips to which organic materials are absorbed which have passed through the first and second wood chip absorbing tanks into the composting tank;
a first return line for transferring sludge from the denitrification tank to the anaerobic tank;
one or more second return lines for transferring sludge from the precipitation tank to the composting tank, anaerobic tank, first and second wood chip absorbing tanks and microbial activation tank, respectively;
a second inflow line for feeding sludge from the air floatation tank to the composting tank;
a waste sludge line for discharging sludge from the precipitation tank;
a liquefied fertilizer collection line for utilizing the resulting material stabilized in the aeration tank as a liquefied fertilizer; and
a third inflow line for feeding short-chain organic acids from the second wood chip absorbing tank into the denitrification tank.

14. The apparatus according to claim 12, wherein the amount of food waste fed into the first wood chip absorbing tank is in the range of 68 to 70 kg/l per day.

15. The apparatus according to claim 12, wherein hydraulic retention time (HRT) of food waste in each of the first and second wood chip absorbing tanks is 20 to 24 hours.

16. The apparatus according to claim 12, wherein the suspended particles in the air floatation tank are transferred to the composting tank through a return line, and the remaining supernatant containing organic materials is transferred to the anaerobic tank.

17. The apparatus according to claim 12, wherein the aerobic microorganisms activated in the microbial activation tank are transferred to the aeration tank, and the sludge is fed to the microbial activation tank from the precipitation tank through a return line.

18. The apparatus according to claim 12, wherein the aeration tank is divided into a plurality of compartments by means of a plurality of membranes, and wherein an amount of dissolved oxygen (DO) in each compartment is differentially maintained in a graduated manner.

19. The apparatus according to claim 18, wherein the aeration tank is divided into three compartments, wherein the dissolved oxygen (DO) of a first, second, and third compartment are maintained in the range of 0.5 to 1.0 mg/l, 0.2 to 0.5 mg/l, and 0.1 mg/l or below, respectively.

20. The apparatus according to claim 12, wherein the denitrification tank is fed the resulting material containing short-chain amino acids passed through the first and second wood chip absorbing tanks.
